(19) United States
(12) Patent Application Publication
(10) Pub. No.: US 2024/0252357 A1
(43) Pub. Date: Aug. 1, 2024

(54) VISUAL PROTECTION DEVICE FOR TASK FORCES

(71) Applicant: RUBI GLAS GMBH, Michendorf OT Wilhelmshorst (DE)

(72) Inventor: Martin Manfred JAHR, Michendorf OT Wilhelmshorst (DE)

(21) Appl. No.: 18/565,915

(22) PCT Filed: May 31, 2022
(86) PCT No.: PCT/DE2022/100408

§ 371 Date: Nov. 30, 2023

(30) Foreign Application Priority Data
May 31, 2021 (DE) 10 2021 113 990.2

(51) Int. Cl. A61F 9/02; G02C 7/10

(52) U.S. Cl. CPC A61F 9/022; G02C 7/104

(57) ABSTRACT

The invention relates to a visual protection device (1) for task forces. According to the invention, in order to reduce the effects of potentially traumatic events on task forces, at least one viewing lens (6) of the visual protection device (1) is designed to reflect and/or absorb light with wavelengths of more than 640 nm, more than 600 nm, more than 560 nm or more than 550 nm more strongly than light in the shorter wavelength range, wherein the shorter wavelength range extends up to wavelengths of 640 nm, 600 nm, 560 nm or 550 nm.

### BACKGROUND OF THE INVENTION

### Field of the Invention

The present invention relates to the field of visual protection devices, in particular visual protection devices for persons and in particular for task forces, such as firefighters, police officers, emergency responders, disaster relief forces, soldiers and those involved in accidents and those who witness them.

### Background Art

Visual protection devices, for example protective goggles or face masks, for example face shields or breathing or oxygen masks, with viewing windows, are used by persons or operational task forces, for example to protect their eyes from harmful influences. The harmful influences may consist of dust or splinters, but also smoke or harmful gases in the case of breathing or oxygen masks.

Those involved in accidents and those who witness them as well as operational task forces are often confronted with seriously injured or even mutilated people. The images of these injuries and mutilations become imprinted in the memories of the task forces and often trigger psychological problems, such as post-traumatic stress disorder.

### SUMMARY OF THE INVENTION

The object of the invention is to reduce the negative psychological effects of situations in which people and in particular task forces are confronted with injured and/or mutilated people.

The object is achieved according to the invention by a visual protection device which has at least one viewing lens, wherein the viewing lens is designed to reflect and/or absorb light with wavelengths of more than 640 nm, more than 600 nm, more than 560 nm or more than 550 nm more strongly than light in the shorter wavelength range, wherein the shorter wavelength range extends up to the wavelength 640 nm, 600 nm, 560 nm or 550 nm. The filter range comprises red light.

Due to the fact that the visual protection device reflects and/or absorbs red light, i.e. filters it out of the visible spectrum, the person perceives this color to a lesser extent or no longer perceives it at all. Red ranges in the person's field of vision are then substantially perceived as dark or black.

Colors and experiences associated with them are imprinted to varying degrees. This effect is particularly strong, if not the strongest, with the color red, so that operational situations in which seriously injured and even dead people need to be treated and/or recovered remain in the memory for a particularly long time and have to be processed again and again. Injured or mutilated people often bleed heavily, with injured flesh and the visible internal organs of these injured or mutilated people also appearing red or reddish in color, for example due to bleeding.

As the visual protection device according to the invention filters out the color red, red areas in the person's field of vision, i.e. blood, injured flesh, internal organs or severed limbs of injured or mutilated people, are less strongly imprinted, so that such situations are less stressful for the person and such traumatizing experiences can be processed more quickly.

The visual protection device according to the invention does not pose a danger, for example when recognizing hazard symbols and traffic signs, as these can be clearly identified by their shape and the corresponding pictograms. In addition, hazard symbols are often displayed in the color orange, which may not be filtered out by the visual protection device according to the invention.

Advantageously, the visual protection device according to the invention is also suitable for persons who are accidentally confronted with injured and dead people. For example, a corresponding visual protection device can be provided in first-aid kits.

The solution can be further improved by various embodiments, each of which is advantageous in its own way and, unless otherwise stated, can be combined with one another as desired. These embodiments and their associated advantages are discussed in the following.

According to a first embodiment the viewing lens is designed to substantially completely reflect and/or absorb red light, i.e. filter it out. Substantially, this can mean that at least 70%, at least 80%, at least 90% or even at least 95% of the light is filtered out, i.e. reflected or absorbed, possibly even so much that it can no longer be perceived at all by the person wearing the visual protection device.

An advantage of this embodiment can be that potentially traumatic impressions make less of an impact.

As already explained, the viewing lens is designed to reflect and/or absorb, i.e. filter out, light with wavelengths of more than 640 nm, more than 600 nm, more than 560 nm or more than 550 nm more strongly than light in the shorter wavelength range. A filter range, in which the light is reflected and/or absorbed by the viewing lens therefore begins at one of the said wavelengths, wherein this wavelength can then be described as a lower or first wavelength value.

Due to the fact that the viewing lens is designed to filter out light with wavelengths of at least 640 nm, at least 600 nm, at least 560 nm or at least 550 nm, it can be an advantage that red light is not only filtered out according to the color spectrum. Filtering out the red light according to the color spectrum already significantly reduces the imprint of potentially traumatic impressions. However, according to the color spectrum, red light is perceived by the human eye in particular by so-called L cones with a maximum absorption typically in a wavelength of light of approximately 563 nm, which corresponds to the color yellow-green in the color spectrum. Excitation of the L cones with light that has shorter wavelengths than light of the color red of the spectrum can result in associations with traumatic impressions despite the different color, because the same cones are excited by the red light according to the color spectrum and light with a wavelength of about 563 nm. In the context of this disclosure, light of any wavelength that excites the L cones of the human eye or at least light having a wavelength greater than and/or equal to the light with the wavelength at the absorption maximum of the L cones may be referred to as red light. In particular, light with wavelengths in the specified filter range can be referred to as red light.

According to a further embodiment, the viewing lens is designed to reflect and/or absorb the light with wavelengths of up to 780 nm or 800 nm more strongly than light of at least one other color and/or also light with a longer wavelength. A filter range in which light is reflected and/or absorbed by the viewing lens can therefore end at one of the said wavelengths, wherein this wavelength can then be denoted as an upper or second wavelength value.

As the viewing lens is designed to filter out light with wavelengths of at most 780 nm or at most 800 nm and in particular if the viewing lens has no structures or elements, i.e. coatings, which reflect light above the said wavelength, infrared light can still get through to the emergency worker, which enables detailed perception of the surroundings by the emergency worker in the event of fire for example. In particular, heat radiation from heat sources, for example a fire, can be sensed.

According to a particularly preferred embodiment of the invention, the viewing lens has a coating in order to provide the viewing lens with an edge filter. Preferably, blocking is provided in the range of 640 to 780 nm.

According to a further embodiment, the visual protection device for filtering light has a bandstop or notch filter by which the light to be filtered out, i.e. light that is not meant to reach the eyes of the task force, is filtered out or blocked, i.e. reflected and/or absorbed.

According to a further embodiment, the visual protection device for filtering has a band pass filter, which only lets through light which is intended to reach the eyes of the task force.

An advantage of these embodiments can be that light can be filtered out or let through specifically according to the wavelength range.

Alternatively, the viewing lens can be designed according to a further embodiment to also filter out and in particular reflect infrared light, whereby the task force can be protected from heat radiation.

According to a further embodiment, the visual protection device is a face mask, wherein at least a portion of a viewing window of the face mask is formed by the viewing lens. In particular, the portion can be a portion of the viewing window through which a person wearing the visual protection device looks. Alternatively, the viewing window can be provided with the viewing lens over its whole surface or consist of the viewing lens.

An advantage of this embodiment can be that in addition to protecting against stressful impressions, the emergency worker can also be protected from other harmful influences such as smoke or gas, without the visual protection device having to be worn in addition to the face mask.

According to a further embodiment the viewing lens is, in at least some segments or entirely, shaped or curved to be cylindrical or spherical.

An advantage of this embodiment can be that, depending on the type of viewing lens used, light is filtered particularly effectively, in particular when it is incidental perpendicular to the viewing lens. Perpendicular can mean that light is incidental to the surface of the viewing lens at an angle of 90°. The angle can deviate by up to ±5°, up to ±10° or up to ±15° from the 90° angle, in order to still be considered perpendicular incidental light within the context of this disclosure.

For example, the visual protection device can have a predetermined viewing direction, in which a person wearing the visual protection device looks through it. The viewing lens may, in at least some segments or entirely, be shaped or curved to be cylindrical or spherical so that the direction of vision is perpendicular to the outside of the portion of the viewing lens through which the person looks. The angle between the viewing direction and the outside can deviate by up to ±5°, up to ±10° or up to ±15° or even up to 25° from the angle 90°, in order to still be considered perpendicular within the context of this disclosure.

According to a further embodiment, the visual protection device is a pair of goggles and in particular protective goggles, with at least one viewing pane and for example two viewing panes, wherein the at least one viewing pane is formed at least in part or fully by the viewing lens or the two viewing panes are formed at least in part or fully by a respective viewing lens.

An advantage of this embodiment can be that the visual protection device is easy and flexible to handle.

According to a further embodiment, the protective goggles have a viewing pane holder, wherein the at least one viewing pane or the viewing panes and the viewing pane holder are integrally formed and for example continuously from the same material. The at least one viewing pane can be formed from a viewing lens. The viewing panes can be formed respectively by viewing lenses. The at least one viewing pane and the viewing pane holder can be formed from the material of the viewing lens. The viewing panes and the viewing pane holder can be made from the material of the viewing lens.

An advantage of this embodiment can be that the visual protection device is easier to manufacture.

According to a further embodiment, the viewing lens can have a reflective and/or absorbent coating for reflecting and/or absorbing the light.

An advantage of this embodiment can be that coatings with the desired properties can be manufactured easily and economically. In particular, the coatings can be applied to commercially available visual protection devices, either while they are being manufactured or even afterwards, thereby reducing the costs of developing and manufacturing the visual protection devices.

Alternatively, or in addition, the material of the viewing lens itself can be designed to reflect and/or absorb light of the said wavelengths.

An advantage of this embodiment can be that the viewing lens with the desired properties can be manufactured easily and economically, for example by injection molding.

According to a further embodiment, the viewing lens is made substantially of optical glass, for example mineral glass, or from transparent plastic, for example polycarbonate, and optionally provided with a reflective and/or absorbent coating for reflecting and/or absorbing the light in the specified wavelength range.

The red light can include light in wavelengths reflecting arterial and venous blood. The red light can correspond to or have the color blood red. The color blood red can be the RAL color 3020 or 3003.

The reflected and/or absorbed wavelength range can include the typical colors of arterial and venous blood or the color blood red and optionally other wavelengths which belong to the color red according to the color spectrum or which are significantly absorbed by L cones.

In the context of this disclosure a color can be a color of the visible light spectrum.

### BRIEF DESCRIPTION OF THE DRAWINGS

For a better understanding of the present invention reference is made to the drawings below. The latter merely show exemplary embodiments of the subject-matter of the invention. Features of these exemplary embodiments can be combined independently of one another.

In the Figures and the associated description the same reference signs are used to denote identical or identically acting parts.

FIG. 1 shows a transmission curve of an exemplary embodiment of the viewing lens of the visual protection device according to the invention,

FIG. 2 shows an exemplary embodiment of the visual protection device according to the invention,

FIG. 3 shows a further exemplary embodiment of the visual protection device according to the invention,

FIG. 4 shows a further exemplary embodiment of the visual protection device according to the invention,

FIG. 5 shows a further exemplary embodiment of the visual protection device according to the invention,

FIG. 6 shows a transmission curve of a preferred embodiment of the viewing lens with edge filter with blocking in the range of 640-780 nm, and

FIG. 7 shows various transmission curves of the viewing lens according to FIG. 6 to illustrate the angular dependence.

### DETAILED DESCRIPTION OF THE PREFERRED EMBODIMENT

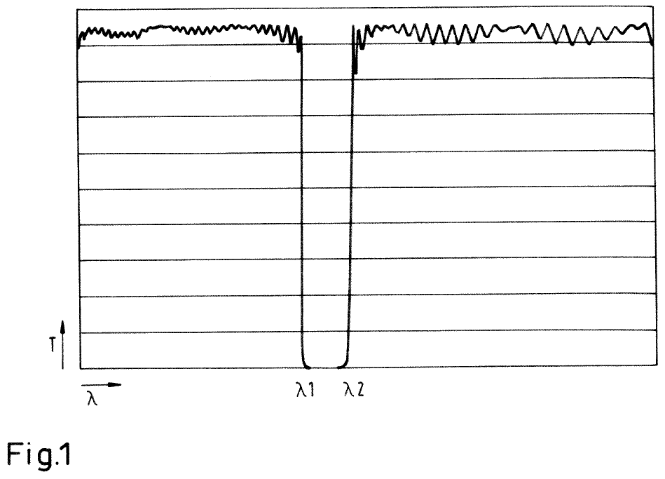

FIG. 1 schematically shows a transmission curve of an exemplary embodiment of the viewing lens of the visual protection device according to the invention, wherein the transmissivity T of the viewing lens is plotted against the wavelength λ of the light. For example, values of transmissivity T from 0% to 100% are plotted on the vertical axis. A wavelength range can be plotted on the horizontal axis of wavelength λ which at least partially covers the visible spectrum and can optionally extend into the infrared range.

Light with wavelengths below a predetermined first wavelength value λ1 is transmitted substantially unhindered by the viewing lens, so that, for example, more than 70%, more than 80% or even more than 90% of this light is transmitted.

From and above the first wavelength value λ1 the transmission of light is significantly lower, so that, for example, less than 30%, less than 20%, less than 10% or even less than 5% of this light is transmitted. The light can be reflected and/or absorbed by the viewing lens in order to reduce or even prevent its transmission through the viewing lens. Light with wavelengths longer than or equal to the first wavelength values λ1 could be partially filtered out by the viewing lens, i.e. by up to 70%, up to 80%, up to 90% or even up to 95%, or even completely, i.e. substantially by up to 100%.

The first wavelength value λ1 can be a wavelength which corresponds to the shortest wavelength of light, which is considered to be red light according to the color spectrum or according to this disclosure.

For example, the first wavelength value λ1 can be a wavelength of 550 nm, 560 nm, 600 nm or 640 nm. Light with a wavelength above one of these wavelengths can be referred to as red light within the context of this disclosure. Alternatively, in the context of this disclosure, this light can also be referred to as longwave light, which is strongly imprinted and/or excites the L cones of the human eye more strongly than light of other wavelengths.

The light filtered out can include or consist of the red component of light reflected by arterial and venous blood. The light filtered out can include light of the color blood red. The color blood red can be the RAL color 3020 or 3003.

The reflected and/or absorbed wavelength range can include the colors of arterial and venous blood or the color blood red and optionally other wavelengths that belong to the color red according to the color spectrum or according to this disclosure.

Optionally, the viewing lens can be designed to filter out light only up to a second predetermined wavelength value λ2. The second wavelength value λ2 is preferably greater than the first wavelength value λ1. For example, the second wavelength value λ2 can correspond to the upper end of the wavelength range of light that belongs to the color red according to the color spectrum or forms the transition to infrared. For example, the second wavelength value λ2 is a wavelength of 780 nm or 800 nm.

Light with wavelengths above the second wavelength value λ2 can pass substantially unhindered through the viewing lens, so that for example more than 70%, more than 80% or even more than 90% of this light is transmitted by the viewing lens. Alternatively, the viewing lens can reflect light with wavelengths above the second wavelength value λ2, so that more than 70%, more than 80% or even more than 90% of this light is reflected.

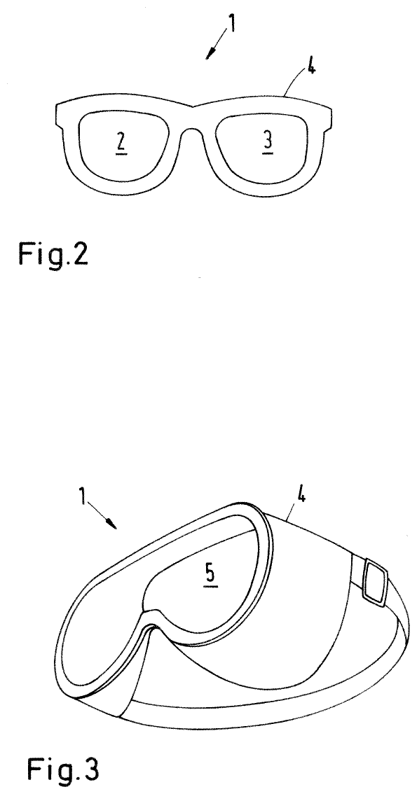

FIG. 2 shows a first exemplary embodiment of the visual protection device 1 according to the invention in a schematic front view. The visual protection device 1 is represented by way of example as glasses with two viewing panes 2, 3, wherein the glasses further comprise a viewing pane holder 4 to which the viewing panes 2, 3 are securely attached. For example, the viewing panes 2, 3 are attached to the viewing pane holder 4 in a form-fit, force-fit or material-bonded manner. Alternatively, the viewing panes 2, 3 and the viewing pane holder 4 can be integrally manufactured, i.e. continuously and from the same material and for example from the material of the viewing lens.

Both viewing panes 2, 3 can each consist, at least partially or completely, of one viewing lens.

The viewing lens can be designed to be cylindrical or spherical as a whole. Alternatively, the viewing lens can comprise a plurality of segments which can be arranged adjacent to or spaced apart from one another, wherein selected ones or all of the segments may be cylindrical or spherical.

FIG. 3 shows a further exemplary embodiment of the visual protection device 1 according to the invention in a schematic perspective view. The same reference signs are used for elements which correspond in form or function to the elements of the previous exemplary embodiment. In the following, for the sake of brevity, only differences from the previous exemplary embodiment are discussed.

The visual protection device 1 of the exemplary embodiment of FIG. 3 is also designed by way of example as a pair of goggles for example and in particular a pair of protective goggles. The protective goggles have only one viewing pane 5, which is dimensioned such that a person wearing this visual protection device 1 can look through the viewing pane 5 with both eyes at the same time. For example, the viewing pane 5 is attached to the viewing pane holder 4 in a form-fit, force-fit or material-bonded manner. Alternatively, and as shown by way of example in FIG. 3, the viewing pane 5 and the viewing pane holder 4 can be integrally manufactured, i.e. continuously and from the same material, and for example from the material of the viewing lens and can be injection molded for example.

The viewing pane 5 can consist at least partially or fully of a viewing lens.

The viewing lens can be designed to be cylindrical or spherical as a whole. Alternatively, the viewing lens can comprise a plurality of segments which can be arranged adjacent to or spaced apart from one another, wherein selected ones or all of the segments may be cylindrical or spherical.

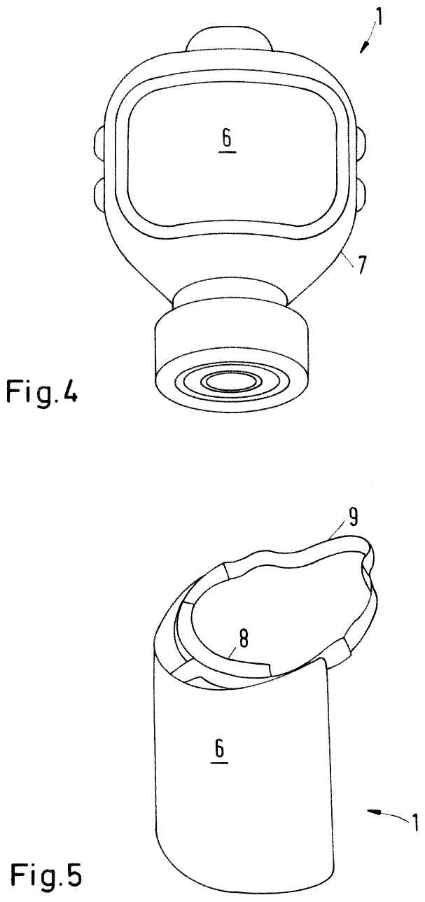

FIG. 4 shows a further exemplary embodiment of the visual protection device 1 according to the invention in a schematic front view. The same reference signs are used for elements which correspond in form or function to the elements of the previous exemplary embodiments. For the sake of brevity, only differences from the previous exemplary embodiments are discussed in the following.

The visual protection device 1 of the exemplary embodiment of FIG. 4 is a face mask which can be in the form of a respiratory mask or an oxygen mask. The face mask has a viewing window 6 which can consist at least partially or fully of the viewing lens. For example, only the portion of the viewing window 6, through which a person wearing the face mask can look when the person is wearing the visual protection device 1, may be provided with the filtering properties of the viewing lens according to the invention. Alternatively, the viewing window 6 may have the filtering properties of the viewing lens according to the invention over its entire surface.

The visual protection device 1 of the exemplary embodiment of FIG. 4 also has a holder 7 for the viewing window 6, wherein the viewing window support 7 can be designed to fit tightly against the face of a person.

The viewing lens or the viewing window 6 can be designed to be cylindrical as a whole. The viewing lens or the viewing window 6 can have a longitudinal axis, about which the viewing lens or the viewing window 6 curves cylindrically. For example, the longitudinal axis of the cylindrically curved viewing lens or the cylindrically curved viewing window 6 can be arranged such that it extends substantially through a plane of vision of a person wearing the visual protection device 1. The eyes of the person can be positioned in the plane of vision. The longitudinal axis can be oriented vertically when a person is wearing the visual protection device 1. For example, the longitudinal axis can extend parallel to the nasal bridge of the person wearing the visual protection device 1. The longitudinal axis can extend parallel to or in a plane of symmetry of the visual protection device 1. The longitudinal axis can extend substantially perpendicular to a viewing direction, in which a person wearing the visual protection device 1 can look through it.

Alternatively, the viewing lens or the viewing window 6 can comprise a plurality of segments which can be arranged adjacent to or spaced apart from one another, wherein selected or all of the segments may be cylindrical or spherical in form.

FIG. 5 shows a further exemplary embodiment of the visual protection device 1 according to the invention in a schematic perspective view. The same reference signs are used for elements which correspond in form or function to the elements of the previous exemplary embodiments. For the sake of brevity, only differences from the previous exemplary embodiments are discussed in the following.

FIG. 5 also shows the visual protection device 1 as a face mask and with the viewing window 6. However, the face mask of the exemplary embodiment 5 is designed without the viewing window holder 7. Rather, the visual protection device 1 of the exemplary embodiment of FIG. 5 has the viewing window 6 and a viewing window support 8 to which the viewing window 6 is attached. The viewing window support 8 can be designed to be attached and detached easily, and where possible also repeatedly, to a helmet or a possibly elastic head band 9. The visual protection device 1 of the exemplary embodiment of FIG. 5 can be referred to as a face shield.

The viewing lens or the viewing window 6 can be designed to be cylindrical as a whole. The viewing lens or the viewing window 6 can have a longitudinal axis, about which the viewing lens or the viewing window 6 curves cylindrically. For example, the longitudinal axis of the cylindrically curved viewing lens or the cylindrically curved viewing window 6 can be arranged such that it extends substantially through a plane of vision of a person wearing the visual protection device 1. The eyes of the person can be positioned in the plane of vision. The longitudinal axis can be oriented vertically when a person is wearing the visual protection device 1. For example, the longitudinal axis can extend parallel to the nasal bridge of the person wearing the visual protection device 1. The longitudinal axis can extend parallel to or in a plane of symmetry of the visual protection device 1. The longitudinal axis can extend substantially perpendicular to a viewing direction, in which a person wearing the visual protection device 1 can look through it.

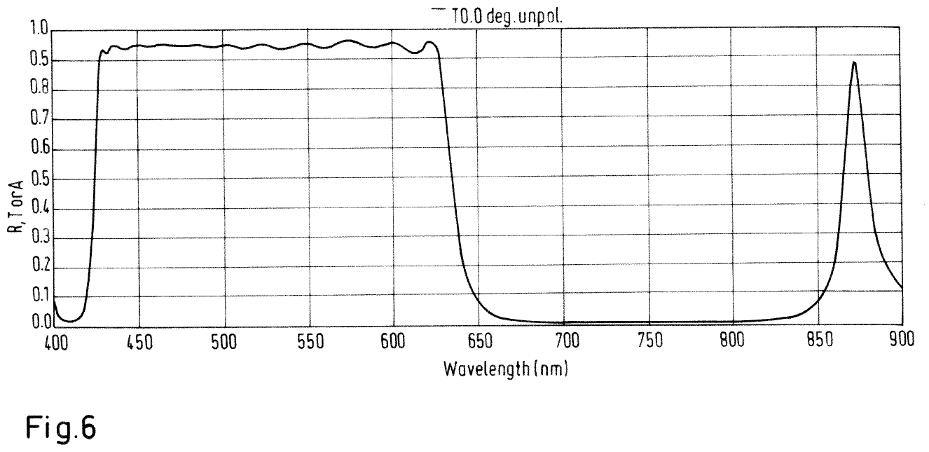

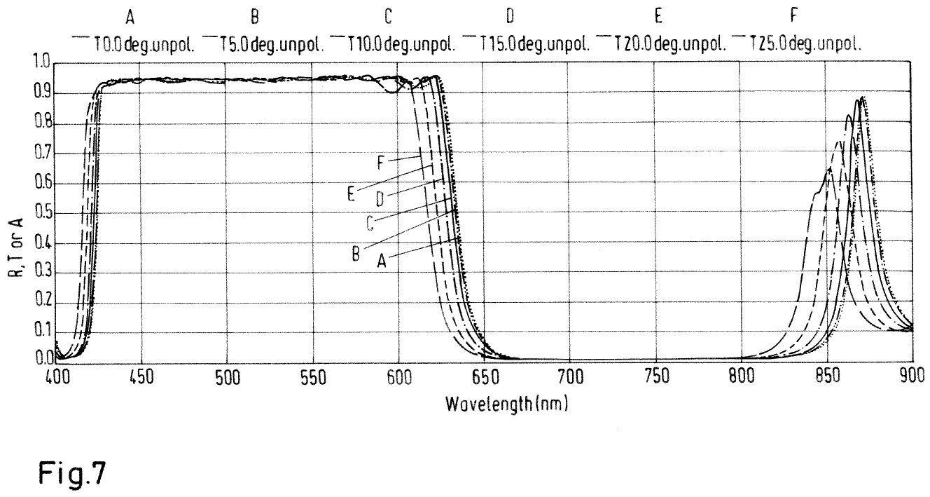

FIGS. 6 and 7 show transmission curves of a preferred embodiment of the protective viewing lens with an edge filter, wherein FIG. 7 shows the angular dependence of the edge filter in the range of 0° (right-angled incidence of light) to 25° (deviation from a right angle) of the incidental light. The transmissivity T of 0.0 to 1.0 of the viewing lens is plotted against the wavelength λ of the light. FIG. 7 shows that in the desired range of 640-780 nm there is almost complete blocking of the viewing lens, which is also not affected by a variation of the angle of incidence. The reference signs A-F represent curves with a 0° to 25° deviation from a right angle at 5° intervals.

### LIST OF REFERENCE SIGNS

1 visual protection device
2, 3, 5 viewing pane
4 viewing pane holder
6 viewing window
7 viewing window holder
8 viewing window support
9 head band
S viewing direction
T transmissivity
λ1 first wavelength value
λ2 second wavelength value
A curve for 0°
B curve for 5°
C curve for 10°
D curve for 15°
E curve for 20°
F curve for 25°

1. A visual protection device for persons and in particular task forces, having at least one viewing lens, wherein the viewing lens is designed to reflect and/or absorb light with wavelengths of more than 640 nm, more than 600 nm, more than 560 nm or more than 550 nm more strongly than light in the shorter wavelength range, wherein the shorter wavelength range extends up to wavelengths of 640 nm, 600 nm, 560 nm or 550 nm.

2. The visual protection device according to claim 1, wherein the viewing lens is designed to substantially fully reflect and/or absorb red light.

3. The visual protection device according to claim 1, wherein the viewing lens is designed to reflect and/or absorb light with wavelengths of up to 780 nm or 800 nm more strongly than light of at least one other color.

4. The visual protection device according to claim 1, wherein the visual protection device is a face mask, wherein at least a portion of a viewing window of the face mask is formed by the viewing lens.

5. The visual protection device according to claim 1, wherein the visual protection device consists of protective goggles with at least one viewing pane, wherein the viewing pane is formed at least partly by a viewing lens.

6. The visual protection device according to claim 5, wherein the protective goggles have a viewing pane holder, wherein the at least one viewing pane and the viewing pane holder are integrally formed, and wherein only the at least one viewing pane or the at least one viewing pane and the viewing pane holder are designed as a viewing lens.

7. The visual protection device according to claim 1, wherein the viewing lens has a reflective and/or absorbent coating for reflecting and/or absorbing the light.

8. The visual protection device according to claim 1, wherein the viewing lens is, in at least some segments or entirely, shaped to be cylindrical or spherical.
